(19) United States
(12) Patent Application Publication
(10) Pub. No.: US 2024/0303595 A1
(43) Pub. Date: Sep. 12, 2024

(54) DEMAND FORECASTING AND INVENTORY MANAGEMENT SYSTEM FOR PHARMACEUTICAL COMPANIES

(71) Applicant: Navneet Kumar, Lexington, MA (US)

(72) Inventor: Navneet Kumar, Lexington, MA (US)

(73) Assignee: Myra EB Systems, Boxborough, MA (US)

(21) Appl. No.: 18/614,705

(22) Filed: Mar. 24, 2024

Related U.S. Application Data
(60) Provisional application No. 63/489,106, filed on Mar. 8, 2023.
(60) Provisional application No. 63/490,247, filed on Mar. 15, 2023.
(60) Provisional application No. 63/490,509, filed on Mar. 15, 2023.

(51) Int. Cl. G06Q 10/087; G06Q 10/0832; G06Q 30/0202

(52) U.S. Cl. CPC G06Q 10/087; G06Q 10/0832; G06Q 30/0202

(57) ABSTRACT

The Demand forecasting combined with an inventory management platform offers robust planning tools customized to the specific needs of pharmaceutical companies. Here, you can create forecasts, measure variance between forecasts, create supply plans, and view the entire supply chain at all stages through an easy-to-use platform that allows you to bring all your data to a centralized location. This system is built explicitly for Pharmaceutical drug substances, drug products, and finished products. The system applies AI/ML to create forecasts and supply plans for clinical trials and commercial distribution.

### FIELD OF INVENTION

The present invention relates to tools for asset management. Mainly, the present invention is directed towards a system and method for planning various aspects of Pharmaceutical Drug products in the supply chain and operations.

### BACKGROUND INFORMATION

In the pharmaceutical and drug industry, predicting or forecasting the amount of medications or vaccines needed is an important part of the industry as not enough medication could result in people not getting the medication they need, while if the need is less than the amount of medication that has been produced, the medication will expire, which results in waste both of the medication and costs. As a result of the current systems, significant medication inventory is written off and the additional costs are passed on to the consumer. Therefore it is necessary for a system to accurately predict the amount of medication that is required to be manufactured so that there is enough medication for the patients while not manufacturing too much that the medication is wasted. The wasted medication also results in significant writeoffs by the company.

The current systems used in the industry require connections with several databases and need help to correctly provide inventory positions for proper planning of various aspects of pharmaceutical drug products in the supply chain and operations. The inventory management platform offers strong planning tools customized to the specific needs of pharmaceutical companies. Here you can view the entire supply chain at all stages through an easy-to-use platform that allows you to bring all your data to a centralized location.

### SUMMARY OF INVENTION

This application claims benefits from provisional patent applications 63/489,106, 63/490,247, and 63/490,509 and these applications are incorporated in their entirety into this application. The invention is for a system to accurately track current amounts of medication when that medication expires and the projected need of drugs, which would allow the pharmaceutical companies to produce enough medication to meet demand while reducing the amount of extra medication that might go to waste.

This application discloses a system and method for planning various aspects of pharmaceutical drug products in the supply chain and operations. The proposed system and method are specifically designed for pharmaceutical products, and currently, multiple systems and databases are required. The proposed system helps in correctly accounting for the Inventory of Pharmaceutical products at various stages (Active Pharmaceutical Ingredient (API) or Drug Substance (DS), Drug Product (DP), and Finished Goods (FGs). The system is capable of handling various constraints that are only applicable to pharmaceutical products. This system will also provide an audit trail and the system will comply with the following FDA regulations. In all our systems, we provide integration with OpenAI ChatGPT 3. System specifically handles a very complex constraint called “Minimum required Shelf Life” (MRSL) and this is required by distributors before they accept any inventory. This constraint is specifically handled which accounts for drug expiry and MRSL needed for drug distribution.

The system would give pharmaceutical and drug companies quick access to past forecasts, to analyze how the system has performed in the past. The analysis would include measurement of the variance between monthly forecasts and the actual need for any given forecast created. Artificial Intelligence and/or Machine Learning engines can create new forecasts based on extrapolation models, analyzing and modifying the models based on past, current, and projected future need and models. The system can use the AI and/or ML engines like an FAQ (Frequently Asked Questions) system to allow the user to interface with the forecast models in a familiar way, allowing the users to ask questions using human language inquires and not complicated methods that might confuse or be overwhelming to senior members of the company. For exampled, ChatGPT-3 provides instant and extensive answers to questions, and trains itself from the chat history, so the system doesn't need to be manually trained.

The first step in the forecasting process is defining the medication or drug and its corresponding SKUs and linking them with the forecasts for different countries. A field sales team for each country provides an SKU-level forecast for a given medication. The field sales team uses various data structures to communicate the field sales forecast, such as Excel, analytical tools, and ERP (Enterprise Resource Planning) to integrate the forecast data into the system. The field sales forecast gets aggregated into a country wide estimate by aggregating all the predictions into a country wide forecast. The country wide forecast is then rolled up to a market level forecast per SKU and the system can connect directly with a product's country wide and market level forecasts. The cycle can be repeated monthly or quarterly, or any period of time selected by a user or the system, for a commercial stage of the manufacturing process. As a result of the many levels of data being aggregated, so much data is created that become very difficult for a person to keep track of based on past data and actuals the forecast for a given month, so the forecasts are generated using AI/ML.

The inventory system aggregates data from various sources, such as current warehouses supplies and ERP systems. The inventory system then aggregates the data by product ID (item numbers, SKUs, or any type of identification), on site inventory, expiration of the medication, quantity of the medication on hand and projected to be needed, and batch identifiers. The inventory system has an MRP (Material Requirements Planning) calculator that processes the data collected from various sources and uses custom-built for the pharmaceutical supply chain parameters in a very user-friendly way. Compared to conventional ERP, the correlation between material, drug substance, and the finished product is very intuitive. The aggregation of LOT Genealogy is the most appreciated feature that is not readily available in the current ERP system, which is needed in product recalls and product investigations.

To accurately predict the future need for a medication, users can quickly access past forecasts. Users can measure the variance between monthly forecasts and actuals and a given forecast while the AI/ML engines recommend the forecast based on extrapolations models. In the future, the capability to switch between models based on user preferences, using customized parameters which can be linked to the forecast for more refined output for the user. Projections of supply plans can be generated based on the company's production capacity for a given medication. The forecasting system also aggregates any excesses and obsolescence (E & O) inventory used for write offs, and the insights around (E & 0) helps commercial companies with the effective use of optimized inventory. This can save big companies millions of dollars of losses for inventory writeoffs.

The inventory system provides notifications to a user of any drop in inventory levels below set targets across product SKU and country levels. Inventory systems provide insights for each SKU and the LOT Geneology, while the AI/ML engines look at the warehouse-level inventory based on the current forecast and can flag any stock outs. The inventory levels for CDMOs (Contract Development and Manufacturing Organization) can be monitored by the platform and linked to various in house and external systems to reflect real-time changes, The shipment system provides the right level of access and notification to get insights on the real-time shipment status, which is done otherwise through heavy email exchanges and excel sheets. The AI/ML engine can classify shipments and sites that get delayed or take longer than expected, and integrating our shipment and inventory modules gives real-time updates to internal and external stakeholders using the platform. Our APIs allow external stakeholders to manage shipments and inventory to co-manage the information in real-time which significantly increases efficiency and allows optimized supply chain management.

Pharmaceuticals and drug companies need to plan for the demand of a particular drug years out at any certain point, which makes the forecasting of the demand so important to the industry. If a company underestimates the demand for a particular drug or medication, there is a potential for loss as potential customers will not have access to the medication. As medication is not the same as a piece of clothing or a food product in that if there is not enough, the customers could suffer and die, as medication for most patients is something they cannot do without or simply purchase an alternative. On the other side, if a company over estimates demand for a drug or medication, millions of dollars could be lost due to the medication being manufactured expiring before they can be shipped and sold to consumers. Therefore there is a need for accurate forecasting to determine the future need for their product.

What makes forecasting so complicated is the amount of variables that need to be factored into the forecast. The system described creates a centralized database for the companies to use to easily see the factors that might hinder the supply line years down the road. The system does this by accessing SKU information, demand information based on countries and regional information provided by sales teams on the ground. The forecasting can be checked each month, so the company can easily access how the forecasting is changed based on updated information provided by the different levels of drug manufacturing, storage, and demand. This allows for a company to measure the variances from month to month and change factors in the manufacturing and storage of medications and the components for medications. Most medications are created from a combination of powders that can be combined in different means to create the medications sold at pharmacies or provided by hospitals to their patients. If a component of a particular medication is set to expire before another component, then the medication cannot be manufactured and distributed.

The system keeps track of all the variables mentioned in the sections above and aggregates them into a central database, which is then provided to the company in the form of an easy to use UI, which allows for different parameters, both individually and in combinations, to be adjusted so the company can see down the road and see how changes in the forecast will affect future supply and demand. The forecasts are created using advanced AI and ML that will run multiple simulations on the data and make the most accurate prediction, and the past forecasts can be stored and accessed by the system to see how past adjustments of parameters affected the current supply and demand for a medication. The UI contains system preferences that can be tailored for a particular customer's needs, so that the pertinent information is easily viewed by the customer, without requiring the customer to change the settings each time they use the system.

The system provides, via the UI, a full visualization of the variances in the forecasts, giving the customer the ability to change current parameters to see how the variances change the forecasts and how to best adjust to the variances. The system allows for the customer to perform an analysis of the root cause of the variances, and view a quick comparison between past and current forecasts, enabling the customer to make the most accurate forecasting for a particular drug or medication. The end goal of the forecasting is the optimization of the supply as mentioned above, as the end goal of 100% supply meeting demand is impossible, but the more accurate the forecast the more likely that the demand and supply will match up as closely as possible.

In the end, the system can give companies the information to accurately predict the amount of a medication needed for a clinical trial, current drug supplies in the case of an epidemic in a particular region or country that might see a surge of the demand for a particular medication, and pipeline management which enables the company to forecast potential demand for medications currently in the research and development stages. The amount of information needed to make the forecast is massive, and each piece of data can affect the predicted supply and demand by a different factor, which adds to the complexity of the forecast. Therefore the system aggregates all the information available to the company, provides the information to the customer in an easy to interpret and manipulate format, and allows for the customer to create and implement a forecast that will best match up supply and demand.

### BRIEF DESCRIPTIONS OF FIGURES

FIG. 1 illustrates a screenshot of the proposed system used for planning various aspects of Pharmaceutical Drug Products in the supply chain and operations.

FIG. 2 illustrates a screenshot of variance analysis, according to an exemplary embodiment.

FIG. 3 illustrates a screenshot for the demand forecast, according to an exemplary embodiment.

FIG. 4 illustrates a screenshot showing inventory management or Inventory information, according to an exemplary embodiment.

FIG. 5 illustrates a screenshot showing a production plan, according to an exemplary embodiment.

FIG. 6 illustrates a screenshot showing an E&O or (Excess and Obsolescence) report, according to an exemplary embodiment.

FIG. 7 shows a ChatGPT Screenshot.

FIG. 8 shows a login interface.

FIG. 9 shows a pharmaceutical information input interface.

FIG. 10 shows a pharmaceutical information input interface.

FIG. 11 shows a pharmaceutical information input interface.

FIG. 12 shows a pharmaceutical information input interface.

FIG. 13 shows a pharmaceutical information input interface.

FIG. 14 shows a pharmaceutical information input interface.

FIG. 15 shows a pharmaceutical information input interface.

FIG. 16 shows a pharmaceutical information input interface.

FIG. 17 shows a pharmaceutical information bar graph.

FIG. 18 shows a pharmaceutical information input interface.

FIG. 19 shows a familial relationship graph.

### DETAILED DESCRIPTION

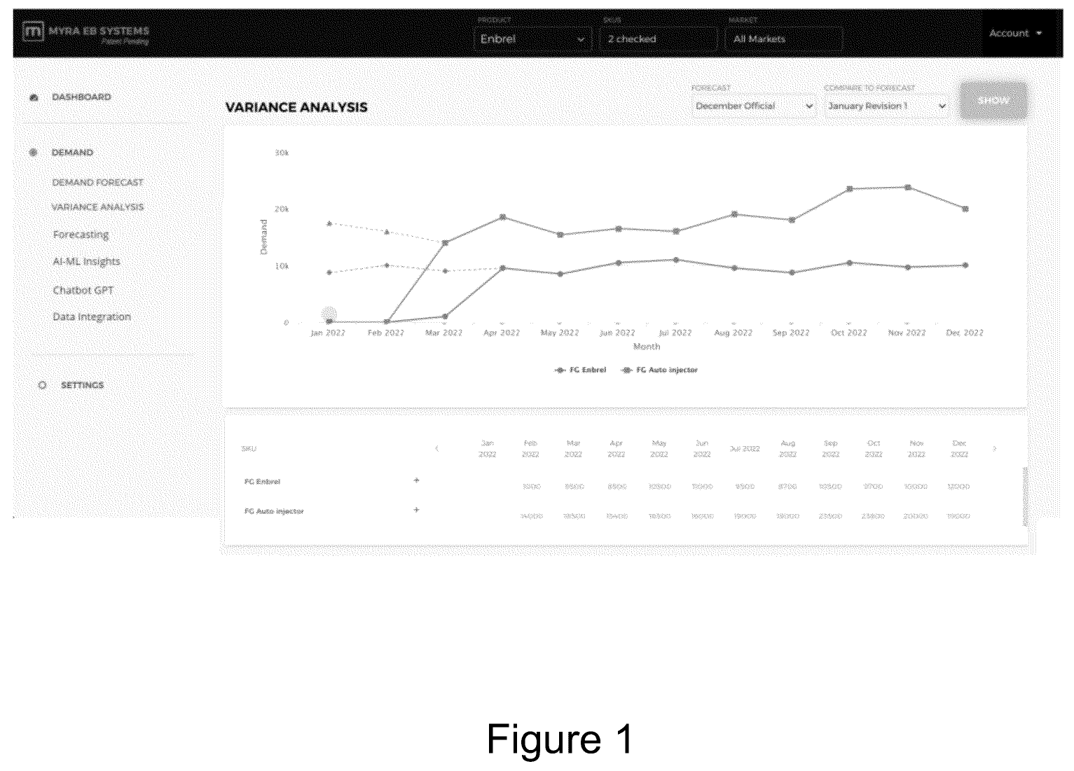

FIG. 1 shows a screenshot of the system's Dashboard or home page that acts as a overview of a Variance Analysis of a particular drug(s). While the screen shot is shown as a preferred example of the system, variations of the screen shown could be used depending on the preferences of the user. The orientation of the options on the side of the screen could be located at any point on the page, in a pull down menu, animated buttons or options, in any order, or any variation that would allow for the same information to be presented to the user. The options shown could be a default setting of the software, which can be customized by the user and saved for future use. This customization would allow for buttons, graphs, images, and any information presented to be added or deleted from this page to only show the information required by the user. The software could also store a plurality of profiles for each user so that a user's preferences could be called up by pressing a button on the page or during a login screen presented when the software is started, allowing for the current user to enter their login information and/or password, select a stored profile, create a new profile, or select the default UI to save time. The profile or preferences could be used for each selectable window, or the profile or preferences could be used for every selectable window available while using the software. The default display could display a line graph as shown, any type of graph or visual representation of the information requested could be used, such as bar graphs, pie charts, 3D graphical representations, raw data, or any other means for displaying information to a user. While this description is applied to FIG. 1, the description would be applicable to every window discussed in the application.

As is shown in FIG. 1, the user has selected the DEMAND button or tab on the left side of the screen, which allows for a user to see the demand for a drug, compound, or part of a drug that is required to analyze the demand of. In this example, the Embrel and Auto Injector demand forecast is displayed to the user. The Official Forecast for December is being compared to the January Revision 1 forecast. The user could select any window of time, either by preselected options in a pull down menu, specific dates/months/years entered by a user, or any other option for selecting a window of time for the user to analyze. In this window, the graph above displays the forecasted data via a line graph so that the trends can be easily identified by the user, while the raw forecast data is given in the window below. As seen in the line graph y-axis, the demand is shown in factors of tens of thousands of units required, but the user could change the y-axis parameters to show more information or less depending on the setting chosen. The x-axis shows months, but any window of time could be used, such as days, weeks, months, years, decades, or any preferred window to easily convey the information to the user. The dotted lines for January, February, March, and April shows the forecasted information found in the January Revision 1 forecast and how it compares to the December Official forecast. There is a difference in the forecasted amounts at the beginning of the year in 2022, but the forecasted amounts line up past March and April in 2022, showing that the forecasted demand matched the actual demand measured. Note that during the shipment and delivery phases, that the information for the expiration for each compound or pharmaceutical needs to be factored into the forecasts.

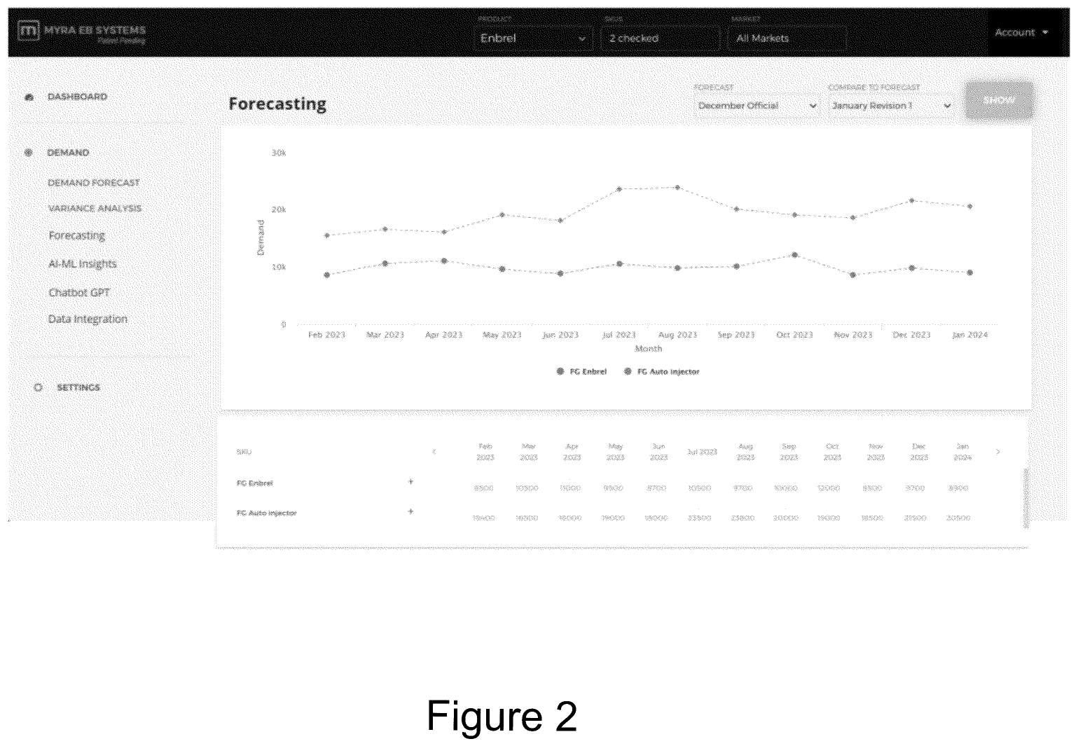

FIG. 2 shows the UI displaying the Demand for the two components that are required to make an Embrel injector, which is the medication and the Auto Injector. At the top of the window are pull down menus allowing the select different parameters for the Demand graph. The pull down menus could be 2D, 3D, virtual, dial, interactive, or any combination of those listed. The first menu allows the user to select which drug they want to see the demand for. The second menu is for selecting the components of the drug, which needs to be at least one, but can be any number. Finally the user can select which markets the user wants to observe, which could be continent, region, country, state, province, village, town, city, or community. The user could receive alerts or notifications in the form of texts, emails, pages, calls, IMs, program pop up or overlays, banners, or any way of alerting a person to incoming information.

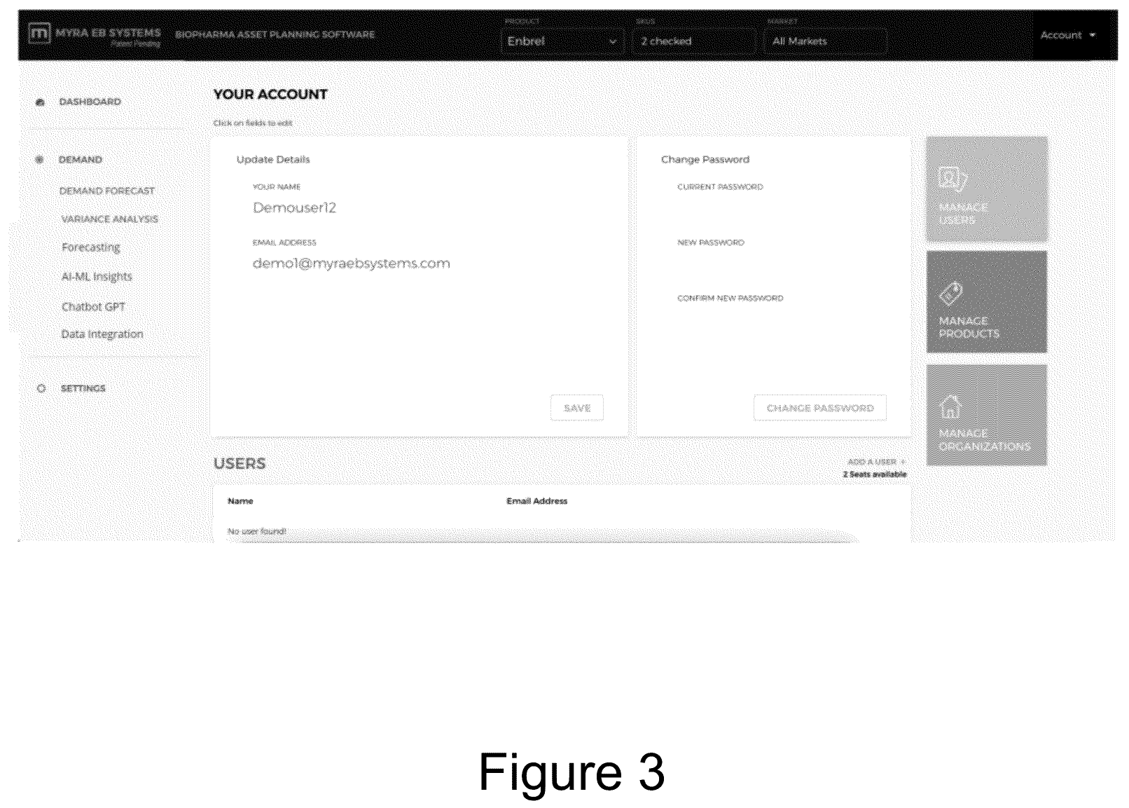

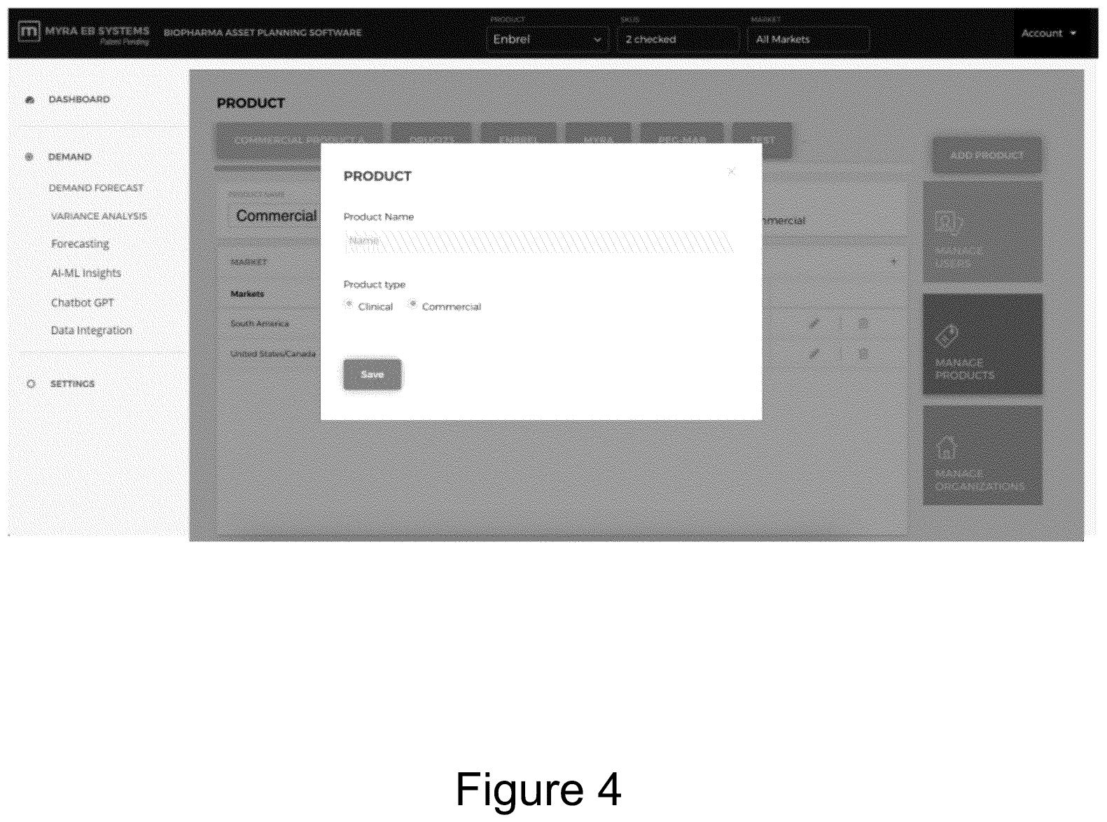

FIG. 3 shows the user account selection screen, which allows for a user to log in and customize the software for their current needs. The user can update their User Name, email address, change or look up their password, manage the other users or profiles for a single user, manage products, and manage organization. The ADD A USER option could be used to invite other users to see the same information that is being displayed to a current user, or it could be used to invite others to work on the current data set being viewed by the user. In FIG. 4 a user selects if the product they want to view the forecast data for is a clinical or commercial drug, and the user is given the option to name the Product, which could be typed in by the user or selected from a drop down menu. The user selects the Save button when the desired information is finished being inputted.

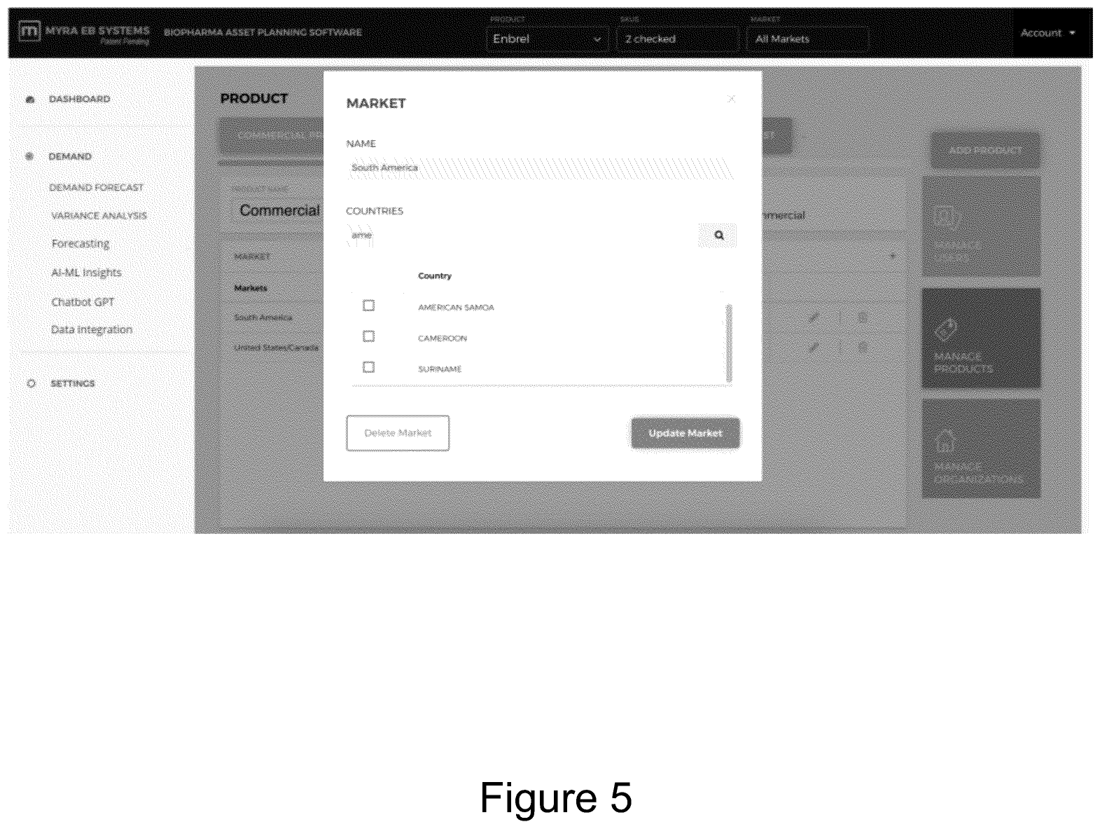

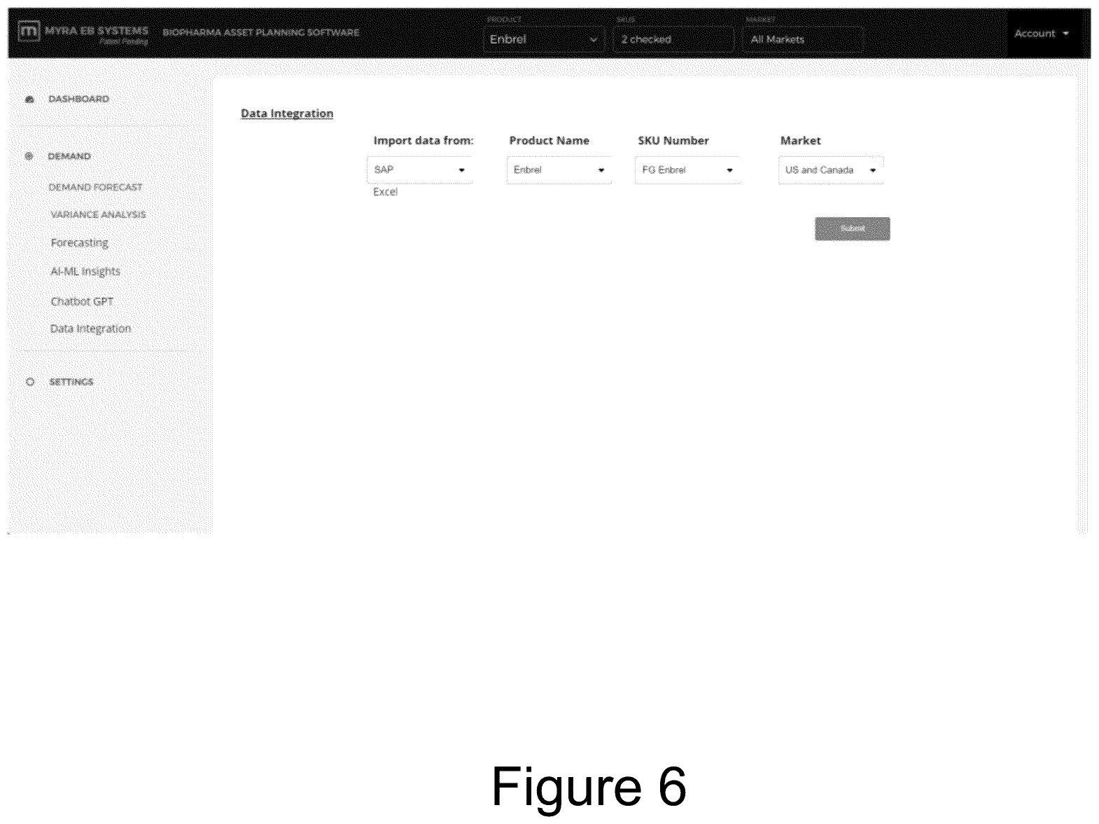

FIG. 5 shows the next option for the user, where they give the market a name, in this case, South America, and then are given the option to select counties that the user wants to analyze the data for. In this example the user can search for a country using an autofill search bar where the options are selected by the user, but the countries could be populated by the continent or region selected by the user which can then be selected by the user, or the system could automatically suggest countries based on the name of the market. The user selects the desired countries by a selection button, allowing for a single or multiple countries to be selected, then the user chooses to update the markets shown in the forecasting data or delete the market. FIG. 6 shows where the software should import the data from, including, but not limited to, SAP data from an Excel spreadsheet, the product name (Embrel), the SKU number (FG Embrel), and the market (US and Canada). While the options presented to the user is shown as only giving the user predefined options to customize, the options presented could be limitless so that the user has unlimited customization options so they can view the data that they need to analyze.

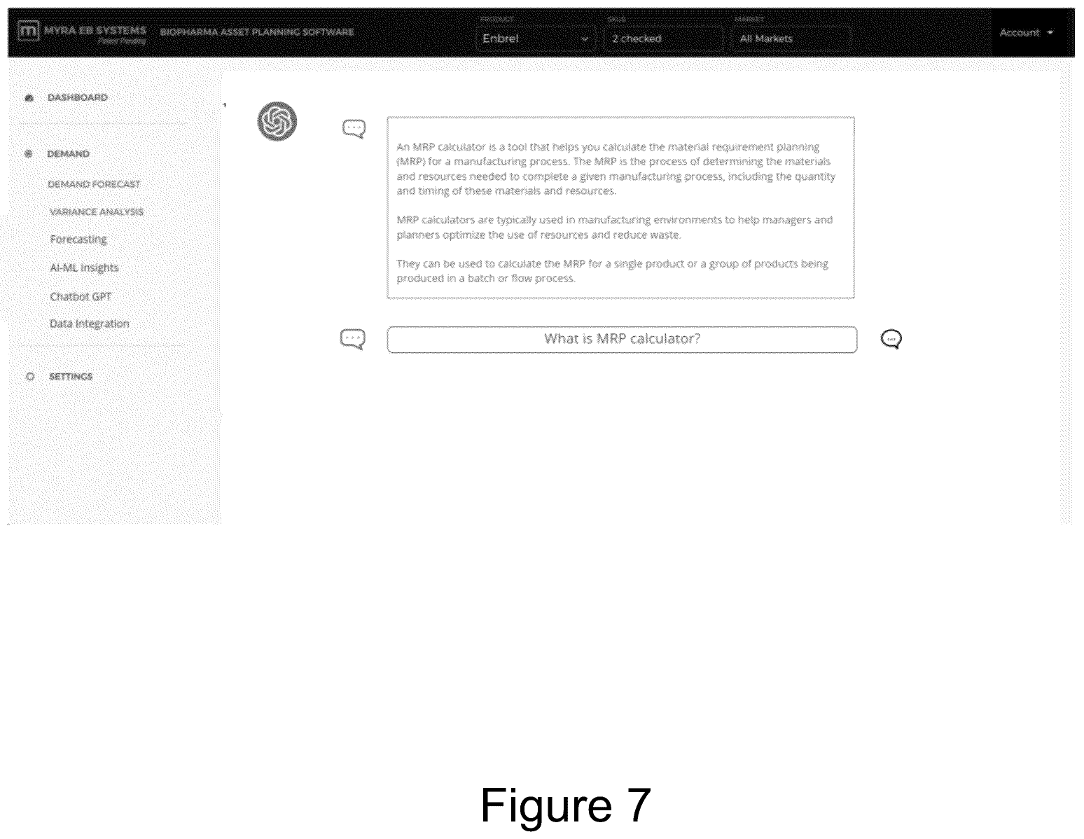

FIG. 7 shows a ChatGPT functionality window being presented to the user. In this window the user can ask the software questions, in this example What is MRP calculator, which the software describes to the user. The ChatGPT functionality would allow for the user to ask questions of the software that gives them answers to any questions they have. The ChatGPT page could also be used by the user to display different forecasts, different forecast windows, different forecasts for drugs or drug components, etc. which the software would then display to the user.

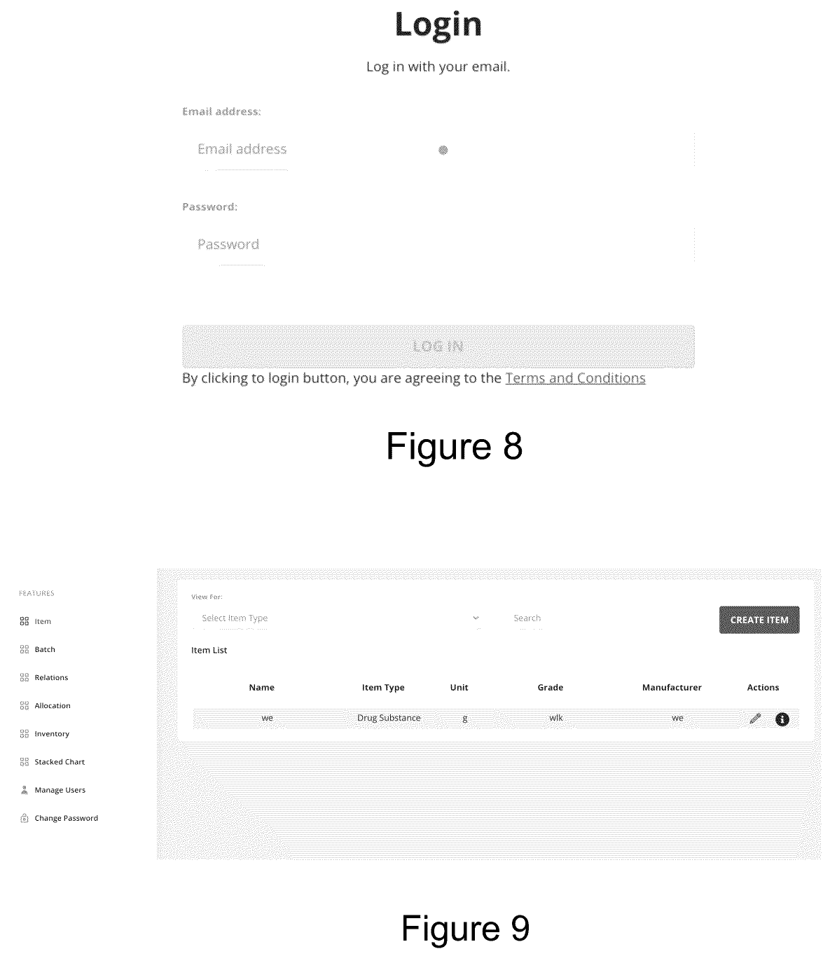

FIG. 8 shows a login interface that the user can input their email address and password. The login could be an email address, user ID, name, or code, while the password could be biometric like a fingerprint, iris, or face scan, voice imprint, password, pin number, or any other identifying mode.

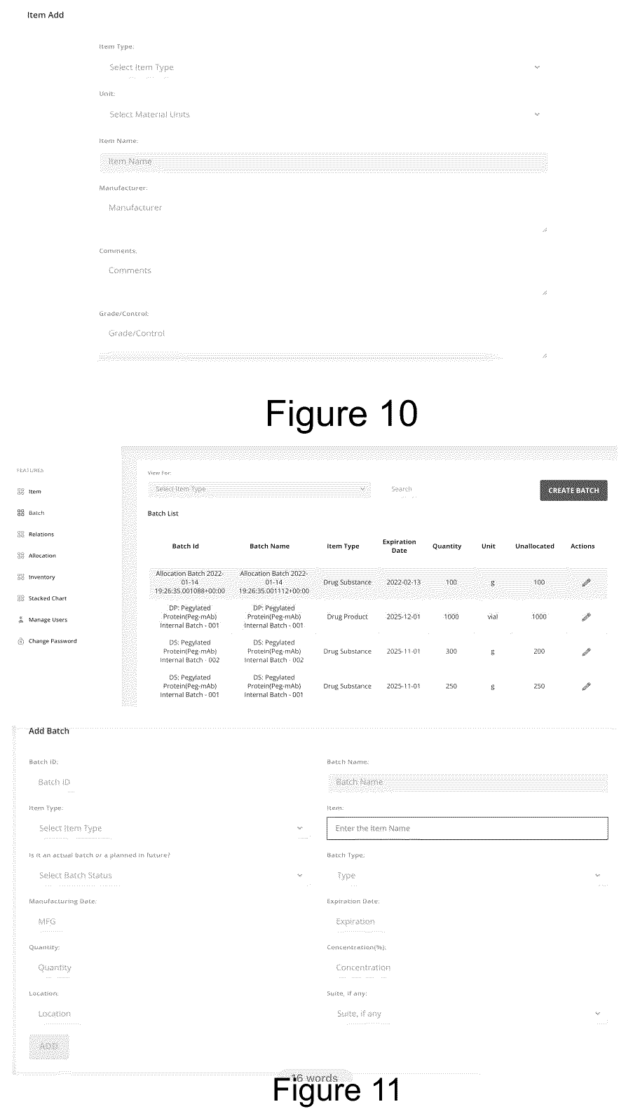

FIG. 9 shows the interface for creating an item to generate a forecast for. In this example, the Item Type is a Drug Substance, but the type could be any type of medication or medical injection system. The user can name the item, select a mass or weight of the drug, assign a Grade, and enter the name of the Manufacturer. This editing is implemented by using an edit button the right of the screen. FIG. 10 shows an interface for entering the details for a drug.

FIG. 10 shows an interface to create batches of the drug entered in the item creating interface. When the user selects the CREATE BATCH option on the interface, it brings up FIG. 11. The second interface could be an overlay, side by side, pip, or a replacement page. FIG. 10 also shows that the Unit can be a container as well as a mass, weight, or volume.

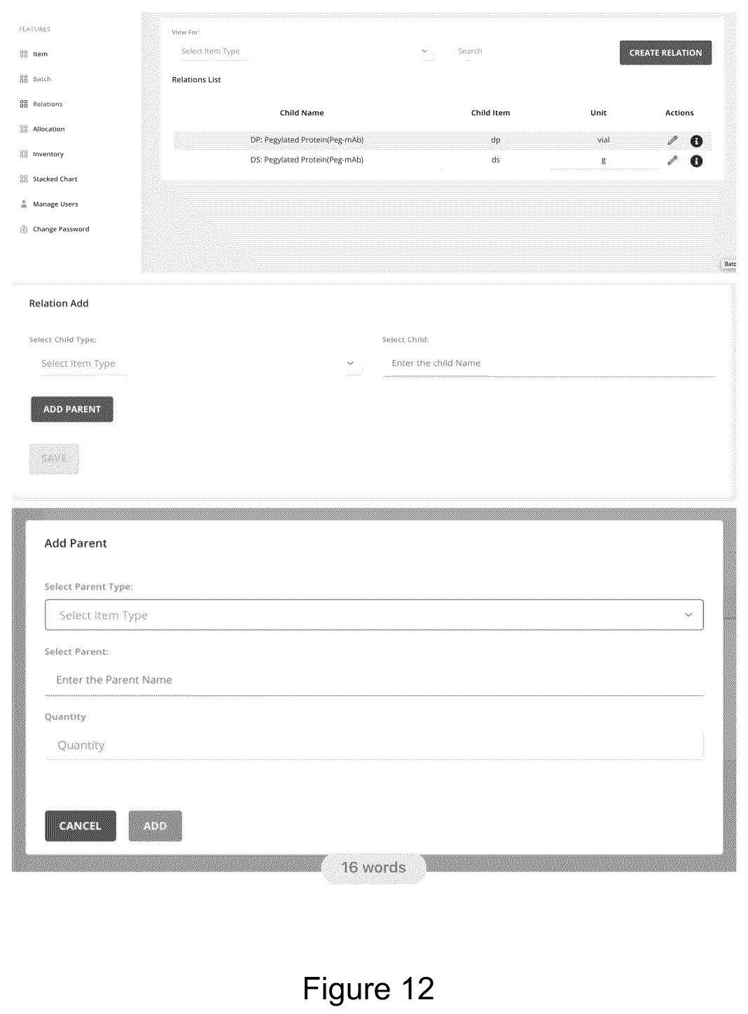

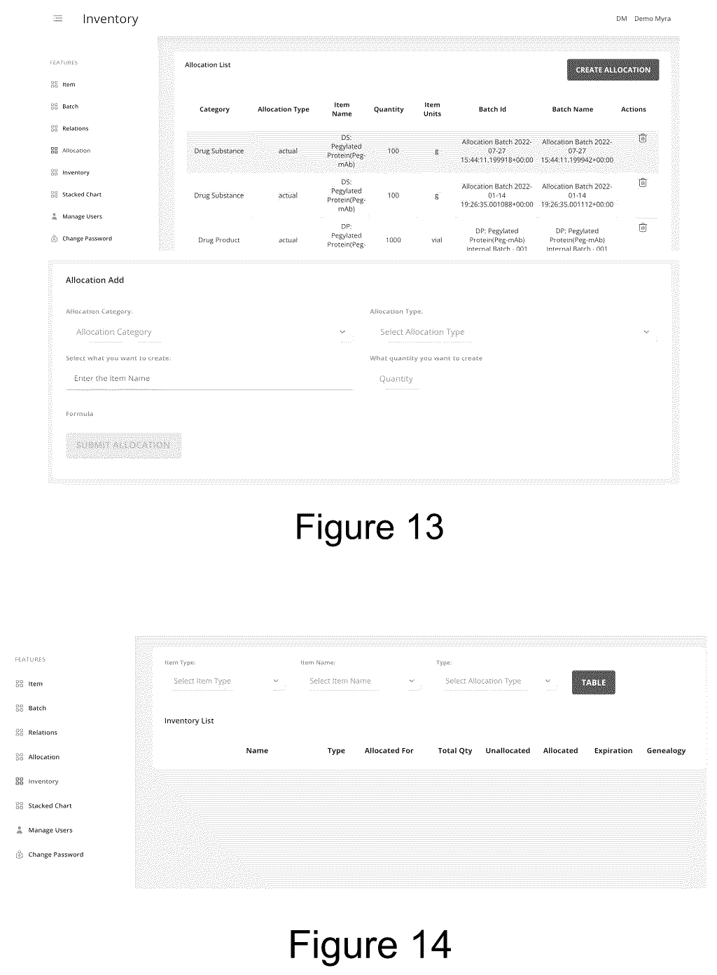

FIG. 12 shows an interface for selecting the relation of one drug to another drug, either as a parent or child. FIG. 13 shows an interface for assigning how the drug is allocated. When the user has inputted all the information they can check the Inventory of the drug as shown in FIG. 14.

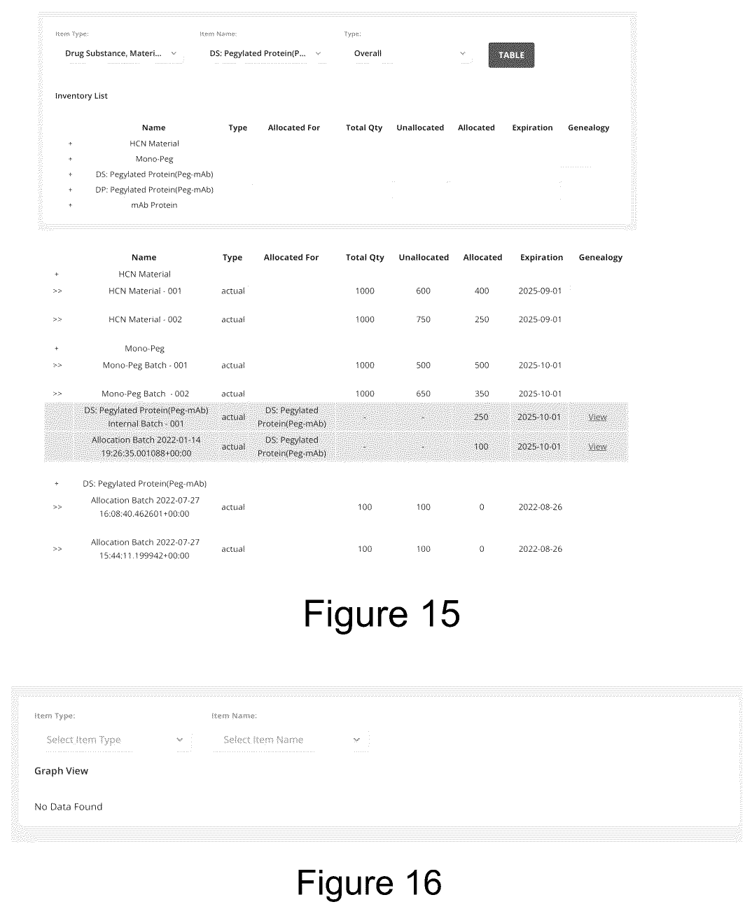

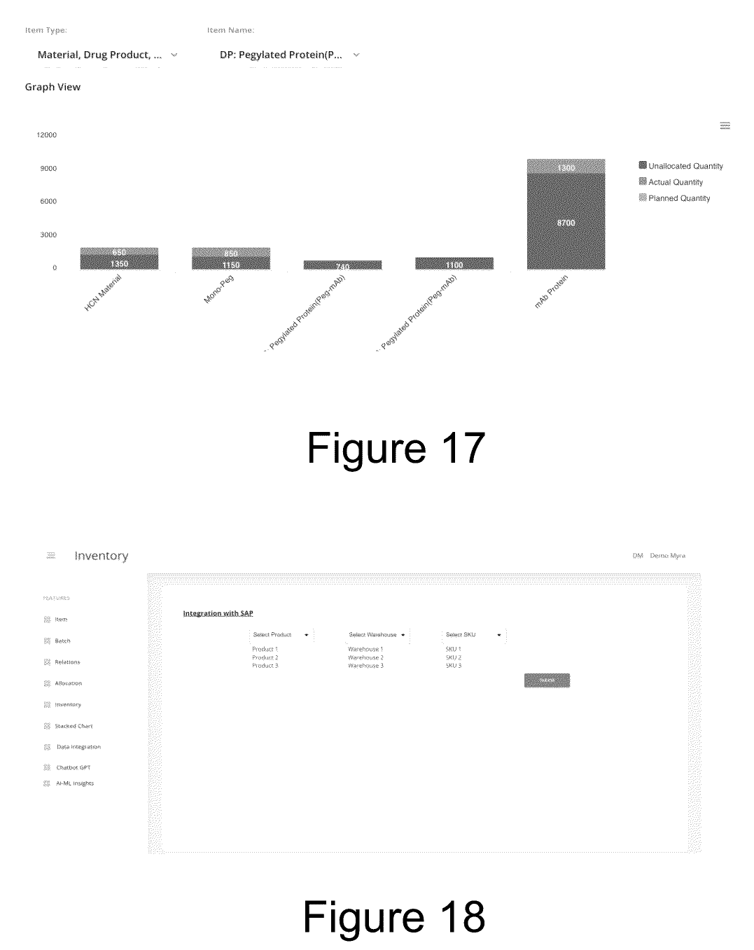

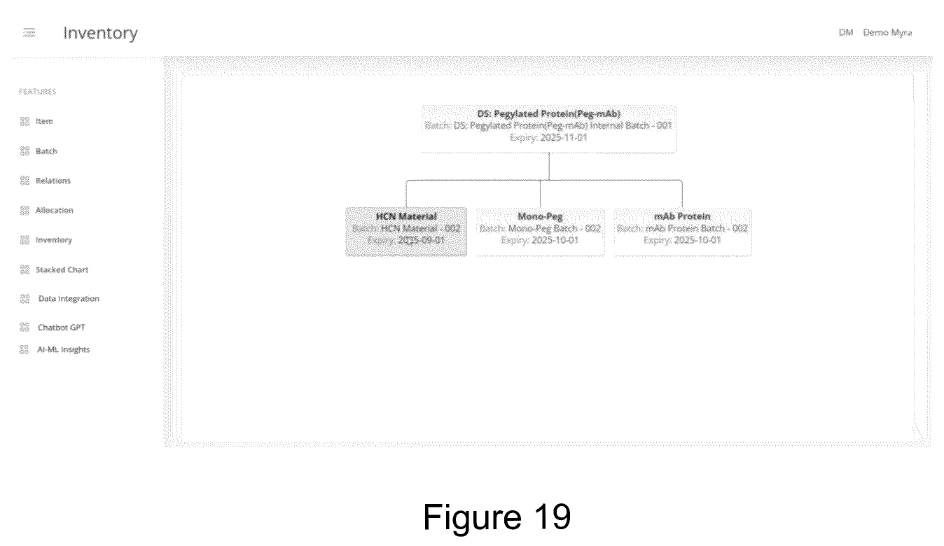

FIG. 15 shows a table view of the drug(s) selected for forecasting while FIG. 16 shows the option to select the graph view. FIG. 17 shows one example of a Graph View, including Unallocated, Actual, and Planned Quantities. FIG. 18 shows an interface where the user can check the inventories for a Product, Warehouse, and SKU. FIG. 19 shows a relation graph for four drugs, one parent, and three children.

The devices mentioned above could be implemented using any type of processor architecture able to execute software including, but not limited to, x86, ENIAC, RISC, Pentium™, and Apple Silicon™. The software could be any type of code that is used to instruct a processor to perform instructions including, but not limited to, Python™, Java™ C+™ FORTRAN, and Assembly. The software could be stored on any type of non-transitory medium including, but not limited to, RAM, ROM, Flash Memory, SD cards, solid stated drives, spinning platter storage devices, Punch Cards, Piano Player Reels, Hard Drives, and physical servers.

We claim:

1. A system for forecasting demand for pharmaceuticals based on inventory, comprising:
an interface for interpreting a user query for a forecast of a pharmaceutical product for a specific time window, and processing the query to instruct the system to aggregate drug inventory information based the inventory of a pharmaceutical supply chain component;
the system uses AI and ML and special connection to the databases and generates forecasts for the commercial and clinical pharmaceutical products, this may require connections with aggregated drug inventory for the each point in the supply chains, and uses previous inventory data and previous forecast data to compare the accuracies of the previous forecasts, wherein:
parameters inputted by the user comprising at least two of the previous inventory data, the previous forecast data, and the comparison data are used along with the current inventory to generate a projected forecast to the user;
an interface to allow for the user to select different compounds, manufactured pharmaceuticals, and delivery methods and change parameters to determine how the forecast changes based on the parameter changes and display the changes to the user.

2. A system for forecasting demand for pharmaceuticals based on inventory as claimed in claim 1, further comprising:
wherein the query comprises a natural language question.

3. A system for forecasting demand for pharmaceuticals based on inventory as claimed in claim 2, further comprising:
wherein the natural language question is processed via an AI or ML system.

4. A system for forecasting demand for pharmaceuticals based on inventory as claimed in claim 3, further comprising:
wherein the processing is performed via a ChatGPT interface.

5. A system for forecasting demand for pharmaceuticals based on inventory as claimed in claim 1, further comprising:
wherein the specific time window is a month or year.

6. A system for forecasting demand for pharmaceuticals based on inventory as claimed in claim 1, further comprising:
wherein the changes are displayed via a visual representation.

7. A system for forecasting demand for pharmaceuticals based on inventory as claimed in claim 6, further comprising:
wherein the visual representation is a natural language text response.

8. A system for forecasting demand for pharmaceuticals based on inventory as claimed in claim 1, further comprising:
wherein the visual representation is a bar graph, line graph, or chart.

9. A system for forecasting demand for pharmaceuticals based on inventory as claimed in claim 1, further comprising:
wherein the parameters for the pharmaceuticals stored at the database include at least one of: pharmaceutical expiration, shipping times, and pharmaceutical manufacturing time.
